(19) United States
(12) Patent Application Publication
(10) Pub. No.: US 2021/0171606 A1
(43) Pub. Date: Jun. 10, 2021

(54) RUBBERY, COMPLIANT, AND SUTURABLE COLLAGEN-BASED SCAFFOLDS FOR TISSUE ENGINEERING APPLICATIONS

(71) Applicant: The Johns Hopkins University, Baltimore, MD (US)

(72) Inventor: Anirudha SINGH, Baltimore, MD (US)

(21) Appl. No.: 17/269,037

(22) PCT Filed: Aug. 21, 2019
(86) PCT No.: PCT/US2019/047405

§ 371 Date: Feb. 17, 2021

Related U.S. Application Data
(60) Provisional application No. 62/720,672, filed on Aug. 21, 2018.

(51) Int. Cl. C07K 14/78; A61L 27/22; A61L 27/24; A61L 27/38; A61L 27/50

(52) U.S. Cl. CPC C07K 14/78; A61L 27/222; A61L 27/24; A61L 2430/30; A61L 27/507; A61L 2430/22; A61L 2430/20; A61L 27/3826

(57) ABSTRACT

A collagen-based or gelatin-based formulation that is compliant yet strong and has a high suture-strength value is disclosed. This material is simple to synthesize, behaves like a rubbery material, and is the first type of solely collagen-based compliant material without using elastin. The formulation maintains the strength of collagen, but can be stretched to several times its initial dimension and can be sutured without leaking. The suture retention strength can reach up to 350-grams force. The presently disclosed collagen-based formulation can be used in variety of applications where high strength, compliance, or stretchability is required, such as in urinary tissues, intestinal tissues, heart tissues, and skin.

### BACKGROUND

Biodegradable elastomers that exhibit rubber-like properties are of great importance in bioengineering, particularly in soft tissue engineering, drug delivery, and in vivo sensing. Langer and Vacanti, 1993; Gilbert et al., 2006; Pamigotto et al., 2000; Badylak et al., 2009; and Nakamura et al., 2016. When implanted in the body, these elastomers must perform functions similar to the host tissues. For the biodegradable elastomer to be clinically attractive, the medical implants should match the biological and mechanical properties of the host tissues and allow the growth of neo-tissues with appropriate restoration of functional properties.

In this regard, several elastomers have been proposed. Most of them, however, are derived from materials ranging from elastin-like peptides, elastin-based materials and synthetic polymers. Nakamura et al., 2016; Chang et al., 2002; Sung et al., 1998; Sung et al., 1999; and Bhrany et al., 2008. A biodegradable elastomer that offers the benefits of similarity in both mechanical and biological properties can be advantageous for a broad range of clinically relevant applications, including reconstruction of urinary tissues and blood vessels. Koo et al., 2006; Suzuki et al. 2001; Haag et al., 2012; and Okada et al., 2007. Since these tissues continuously perform in a complex, mechanically dynamic environment, biomaterials used in their reconstructions are required to be compliant and capable of expanding, contracting, and withstanding fluid pressure repetitively while possessing biological properties similar to the host tissues. Koo et al., 2006; Suzuki et al. 2001; Haag et al., 2012; Okada et al., 2007; Sun et al., 2016; and Gilbert et al., 2013.

Collagen is one of the most ubiquitous mammalian proteins and a major component of tissues. Stein et al., 2012; Shimko et al., 2011. Together with elastin, collagen is mainly responsible for structural integrity and mechanical properties of the physiological tissues. While collagen type I primarily contributes to providing structure and ultimate strength, elastin as a hydrophobic protein enables these tissues to readily recover from large deformations. Van der Aa et al., 2011.

Additionally, an evolved mechanism based on a higher order conformation of collagen type III that can reversibly arrange itself similar to a helical spring contributes to the urinary bladder's ability to expand under minimal fluid pressure. McDougal, 1992; Ferriero et al., 2015. Together, these mechanisms provide the urinary bladder with a remarkable ability to expand under minimal stress or force on urine storage and contract quickly to its initial position on urine voiding. Koo et al., 2006; Sun et al., 2016; Gilbert et al., 2013; Van der Aa et al., 2011; McDougal 1992; Ferriero et al., 2015; and Okhunov et al., 2011. The typical deformation in urinary bladder can reach more than 100%, whereas collagen fibers on their own can usually recover from deformations of about 13-17%. Horst et al., 2013; de Kemp et al., 2015.

While tissue-engineering approaches aim to include complex design criteria in scaffolds, necessary steps often are taken to not over-engineer scaffolds while keeping them clinically relevant. Several scaffolds have been investigated for their abilities to reconstruct/regenerate urinary tissues. Many of these scaffolds depend on chemical crosslinking to modulate stiffness and strength. Chemical crosslinking, however, often compromises the scaffold's compliance and reversible stretchability, causing a mechanical mismatch with the target-repair tissue. As a result, the success of these scaffolds remains elusive in pre-clinical and clinical settings.

### SUMMARY

In some aspects, the presently disclosed subject matter provides a composition comprising a modified collagen, including a modified gelatin, wherein an amine of at least one or more lysine or hydroxylysine residues of the collagen or gelatin is covalently bond to a C₂ to C₁₈ substituted or unsubstituted, saturated or unsaturated aliphatic hydrocarbon chain.

In particular aspects, the modified collagen or gelatin has one or more repeating units comprising the following moiety:

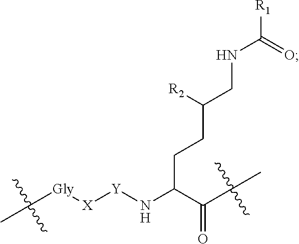

wherein: Gly is glycine; X and Y can be the same or different and are each amino acids; R₁ is selected from the group consisting of —CH₂—CₙH₂ₙ₊ₚ and —CH₂—CH(CH₂C(O)OH)—CH₂—CH═CH—CₙH₂ₙ₊ₚ; wherein n is an integer selected from the group consisting of 2, 3, 4, 5, 6, 7, 8, 9, 10, 11, 12, 13, 14, 15, 16, 17, and 18; and p is 0 or 1; and R₂ is selected from the group consisting of H, —OR₃, —NH₂, C₁-C₄ alkyl, —CF₃, and —COOR₄, wherein R₃ and R₄ are each independently H or C₁-C₄ alkyl. In certain aspects, X is proline. In certain aspects, Y is selected from the group consisting of hydroxyproline, lysine, and hydroxylysine.

In more particular aspects, the presently disclosed composition comprises one or more repeating units comprising the following moiety:

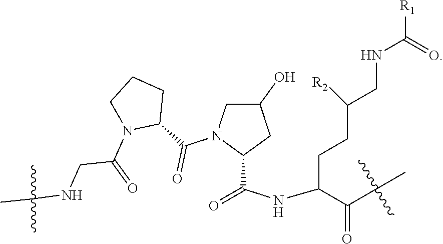

In yet more particular aspects, the presently disclosed composition comprises one or more repeating units selected from the group consisting of:

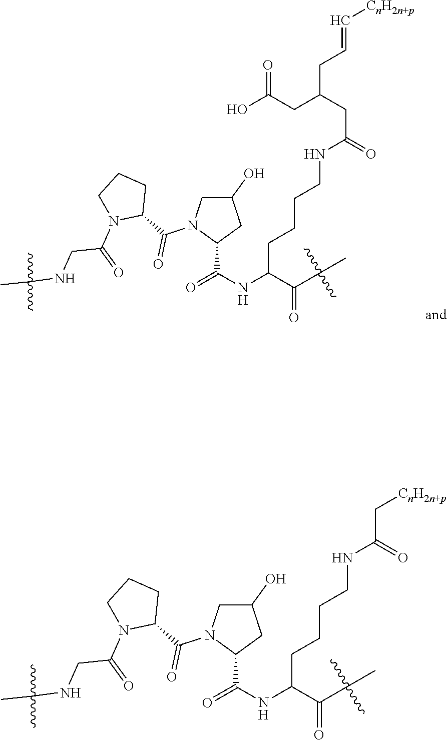

In even yet more particular aspects, p is 1 and n is selected from the group consisting of 9, 10, 11, and 12.

In certain aspects, the composition is about 250% tougher than control collagen. In other aspects, the composition is about 2,500% tougher than control gelatin. In certain aspects, the composition can be reversibly stretched up to about 450% of an initial dimension upon application of about 0.2 MPa force per unit area. In certain aspects, the composition can withstand suture retention loads greater than about 50 gram-force. In yet more certain aspects, the composition can withstand suture retentions loads up to about 350 gram-force.

In other aspects, the presently disclosed subject matter provides a tissue-engineering scaffold comprising the presently disclosed composition. In certain aspects, the scaffold supports in vitro growth of human smooth muscle cells. In particular aspects, the human smooth muscle cells are selected from the group consisting of genitourinary cells, gastrointestinal cells, heart cells, blood vessel cells, and skin cells. In yet other aspects, the presently disclosed subject matter provides an implant comprising the presently disclosed composition.

In other aspects, the presently disclosed subject matter provides a method for preparing a modified collagen or gelatin, the method comprising contacting a collagen or gelatin sample with a solution comprising a C₂-C₁₈ succinic anhydride or C₂-C₁₈ N-hydroxysuccinimide ester and a suitable reagent for a period of time to form a modified collagen. In certain aspects, the suitable reagent is a tertiary amine. In particular aspects, the tertiary amine is 4-(dimethylamino)pyridine (DMAP).

In certain aspects of the presently disclosed subject matter the collagen is selected from the group consisting of collagen I, collagen II, collagen III, and collagen X. In yet more certain aspects, the collagen is collagen type I. In other aspects, the collagen is a gelatin.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, and FIG. 1C show (FIG. 1A) a schematic for steps involved in fabricating collagen discs; (FIG. 1B) an actual laboratory set up with a balloon chamber and vacuum supplies; and (FIG. 1C) a representative molded collagen disc. (Dashed arrow indicates vacuum; solid arrows indicate directions for compression for both white and black arrows);

FIG. 2 is a schematic for modifying collagen with aliphatic groups of varying chain lengths;

FIG. 3A, FIG. 3B, FIG. 3C, FIG. 3D, FIG. 3E, FIG. 3F, FIG. 3G, FIG. 3H, FIG. 3I, FIG. 3J, and FIG. 3K show chemical and structural characterization and properties of representative materials prepared by the presently disclosed subject matter: FIG. 3A, ¹H-NMR, and, FIG. 3B, FTIR-ATR spectra showing modification of collagen with DDSA resulting in resonance peaks corresponding to DDSA at approximately 5.5 and 2.5 ppm and IR bands at approximately 1000 cm⁻¹ and 2650 cm⁻¹, respectively, as indicated in the dotted boxes; FIG. 3C shows the percentage of unreacted amine over time of the reaction at high and low concentrations of modifying agent as determined by TNBS assay; FIG. 3D is a Circular Dichroism (CD) spectra for collagen and modified collagen at pH 3.0 at RT; FIG. 3E shows Differential scanning Calorimetry (DSC) thermograms of modified collagen at different times of reaction to determine its thermal behavior; FIG. 3F shows 2D-X ray diffraction spectra for different materials showing that modified collagen has a collagen-like diffraction peak-pattern although peaks from DDSA apparently are not detectable; FIG. 3G shows TEM images of control-collagen and denatured collagen and modified collagen to determine the ultrastructure of molecules. Swelling ratio of the modified collagen: FIG. 3H, with time at physiological pH condition, FIG. 3I, at different pH values, FIG. 3J, at different temperature and, FIG. 3K, enzymatic degradation behavior overtime at 37° C.;

FIG. 4A, FIG. 4B, FIG. 4C, FIG. 4D, FIG. 4E, FIG. 4F, and FIG. 4G show the viscoelastic properties of the modified material: FIG. 4A, storage modulus, FIG. 4B, Loss modulus and FIG. 4C, Tan(delta) of the modified material with and without vitrification, and its comparison with control-collagen. The modified material derived from vitrified collagen showed flow properties that are retained as shown in heating ramps (1^{st} heating: ramp 1) followed by cooling the material to room temperature and reheating (ramp-2), FIG. 4D, Storage modulus, FIG. 4E, Loss modulus and FIG. 4F, Tan (delta) of the material derived from vitrified collagen. FIG. 4G, Tan (delta) of the modified collagen synthesized from different reaction conditions;

FIG. 5A, FIG. 5B, FIG. 5C, FIG. 5D, FIG. 5E, FIG. 5F, FIG. 5G, FIG. 5H, FIG. 5I and FIG. 5J show the mechanical properties of the fabricated scaffold-Tensile strength, compliance, hysteresis and suturability: FIG. 5A is a hollow scaffold fabricated from modified collagen expands under fluid pressure similar to a rabbit bladder; FIG. 5B shows that the suture retention strength of the modified collagen is similar to the rabbit bladder; FIG. 5C shows the tensile test (stress vs strain) of modified collagen and modified pericardium and their comparison to their control counter parts and a rabbit ureter. FIG. 5D, FIG. 5E, FIG. 5F, FIG. 5G, and FIG. 5H show tensile hysteresis tests for rabbit-ureter, modified collagen, collagen that is crosslinked with gamma-treatment, pericardium and modified pericardium, respectively, and FIG. 5I, and FIG. 5J show suture pull out retention strength values vs strain of the modified collagen and pericardium and its control counterparts;

FIG. 6A and FIG. 6B show that the modified collagen scaffold supports the in vitro cell growth. FIG. 6A shows the morphology of human smooth muscle cells on modified collagen visualized with Actin 488 and NucBlue Microprobe staining. The cells were visualized on the scaffold 28 days post seeding. Stretching pattern of the actin is possibly due to the stretched collagen scaffold in a 48-well plate (20× magnification); and FIG. 6B shows SEM images of cells grown on modified collagen scaffolds with different treatments after 3 days of culture;

FIG. 7A and FIG. 7B, show the in vivo biocompatibility of the modified collagen: FIG. 7A shows gross images of the grafts after 1 week of engraftment. H&E staining of the harvested grafts; FIG. 7B is a collagen control;

FIG. 8A, FIG. 8B, and FIG. 8C show that modified pericardium supports in vivo bladder regeneration: FIG. 8A shows H&E and Trichrome staining of cystectomy control; FIG. 8B is decellularized pericardium; and FIG. 8C is modified decellularized pericardium after 4 weeks of the experiment;

FIG. 9A, FIG. 9B, and FIG. 9C show the viscoelastic properties of the presently disclosed modified gelatin. FIG. 9A is the storage modulus. FIG. 9B is the loss modulus. FIG. 9C is the Tan 6 of the DDSA-modified gelatin and its comparison with control-gelatin; and FIG. 10A, FIG. 10B, and FIG. 10C show the mechanical properties, including tensile strength, compliance, hysteresis, and suturability of the presently disclosed modified gelatin. FIG. TOA is the tensile stress vs strain. FIG. 10B is the suture retention strength of DDSA-modified gelatin and control. FIG. 10C is the tensile hysteresis tests for DDSA-modified gelatin.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Rubbery, Compliant, and Suturable Collagen-Based Scaffolds for Tissue Engineering Applications

Collagen, being one of the most suitable and most available natural biomaterials, is a choice for constructing tissue-engineering scaffolds, mainly due to its biocompositional similarity to physiological tissues. Collagen-scaffolds developed in vitro, however, lack appropriate mechanical properties matching the repair-tissue. While it is possible to enhance the stiffness of the collagen scaffolds by crosslinking, crosslinking often compromises the scaffold's ability to expand or strain under minimal stress, i.e., compliance (also known as inverse of stiffness).

Being an important design criterion, particularly in soft tissue engineering, such as in reconstruction of vasculature and genitourinary system, development of a compliant, yet surgically durable collagen-scaffold is long sought. The presently disclosed subject matter provides, in part, a simple, inexpensive, elastin-free collagen-based material composition that is easy to develop into elastomeric scaffolds that are highly compliant, soft, yet tough, and suturable for potential applications in pre-clinical and clinical settings.

In some embodiments, the presently disclosed modified collagen-based material is approximately 250% tougher than control collagen (or approximately 2,500% tougher than control gelatin) and can be reversibly stretched up to 450% compared to only about 20% for control collagen on application of only 0.2 MPa force per unit area. Furthermore, it can withstand suture retention loads greater than 50 gram-force. The resultant scaffold supports long-term in vitro growth of human smooth muscle cells and is biocompatible in vivo when implanted subcutaneously and in a bladder augmentation model for 4 weeks.

Accordingly, in some embodiments, the presently disclosed subject matter provides a simple, inexpensive, elastin-free collagen-based or gelatin-based material composition for developing elastomeric scaffolds that are highly compliant, soft, yet strong and suturable, and clinically attractive. The presently disclosed scaffolds offer a unique combination of sufficient flexibility, adequate tensile strength, and high suture retention strength values. These physical properties represent the ideal biomechanics desired for tissue engineering of soft tissues. Koo et al., 2006; Gilbert et al., 2013; Kates et al., 2015; Sopko et al, 2015; and Pokrywczynska et al., 2014.

Without wishing to be bound to any one particular theory, it is thought that, in addition to advantageous biological cues present in collagen, mechanical similarities will be highly desirable for eventually creating a biomechanically functional urinary tissue. One object of the presently disclosed subject matter is to develop an elastomeric collagen or gelatin formulation, evaluate the resultant scaffold for its viscoelastic and mechanical properties, and study its biocompatibility both in in vitro and in vivo. It is thought that the presently disclosed material composition and formulation will have an immediate impact in genitourinary, gastrointestinal, and blood vessels-related tissue engineering applications where tissue compliance, i.e., the scaffold's ability to expand or strain under minimal stress, is a key mechanical property.

A. Modified Collagen or Gelatin Compositions

In some embodiments, the presently disclosed subject matter provides a composition comprising a modified collagen or gelatin, wherein an amine of at least one or more lysine or hydroxylysine residues of the collagen or gelatin is covalently bond to a C₂ to C₁₈ substituted or unsubstituted, saturated or unsaturated aliphatic hydrocarbon chain.

Collagen is the main structural protein in the extracellular space in the various connective tissues in animal bodies. At least 28 types of collagen have been identified to date. In general, the types of collagen can be broken down into the following groups according to the structure they form: fibrillar (Type I, II, III, V, XI); non-fibrillar; FACIT (Fibril Associated Collagens with Interrupted Triple Helices) (Type IX, XII, XIV, XIX, XXI); short chain (Type VIII, X); basement membrane (Type IV); multiplexin (Multiple Triple Helix domains with Interruptions) (Type XV, XVIII); MACIT (Membrane Associated Collagens with Interrupted Triple Helices) (Type XIII, XVII); and other (Type VI, VII). The most common types of collagen include: Type I: skin, tendon, vasculature, organs, bone (main component of the organic part of bone); Type II: cartilage (main collagenous component of cartilage); Type III: reticulate (main component of reticular fibers), commonly found alongside type I; Type IV: forms basal lamina, the epithelium-secreted layer of the basement membrane; and Type V: cell surfaces, hair, and placenta. In certain embodiments, the collagen is selected from the group consisting of collagen I, collagen II, collagen III, and collagen X. In particular embodiments, the collagen is collagen I. In other embodiments, the collagen is a gelatin.

In certain embodiments, the modified collagen or gelatin has one or more repeating units comprising the following moiety:

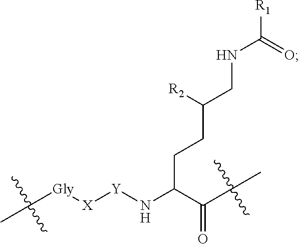

wherein: Gly is glycine; X and Y can be the same or different and are each amino acids; R₁ is selected from the group consisting of: —CH₂—CₙH₂ₙ₊ₚ and —CH₂—CH(CH₂C(O)OH)—CH₂—CH═CH—CₙH₂ₙ₊ₚ; wherein n is an integer selected from the group consisting of 2, 3, 4, 5, 6, 7, 8, 9, 10, 11, 12, 13, 14, 15, 16, 17, and 18; and p is 0 or 1; and R₂ is selected from the group consisting of H, —OR₃, —NH₂, C₁-C₄ alkyl, —CF₃, and —COOR₄, wherein R₃ and R₄ are each independently H or C₁-C₄ alkyl. In certain embodiments, X is proline. In certain embodiments, Y is selected from the group consisting of hydroxyproline, lysine, and hydroxylysine.

In more particular embodiments, the presently disclosed composition comprises one or more repeating units comprising the following moiety:

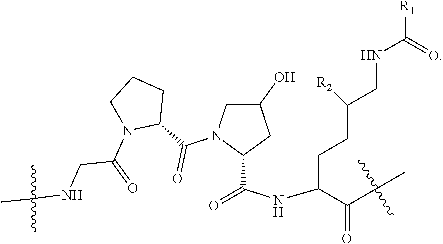

In yet more particular embodiments, the presently disclosed composition comprises one or more repeating units selected from the group consisting of:

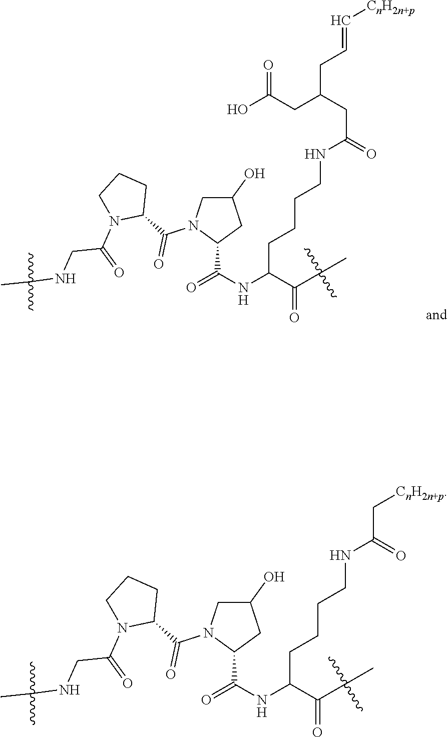

In even yet more particular embodiments, p is 1 and n is selected from the group consisting of 9, 10, 11, and 12.

In some embodiments, the presently disclosed composition is about 250% tougher than control collagen or gelatin, including about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, and 275% tougher than control collagen.

In some embodiments, the presently disclosed composition can be reversibly stretched up to about 450% of an initial dimension upon application of about 0.2 MPa force per unit area, including about 25%, 50%, 75%, 100%, 125%, 150%, 175%, 200%, 225%, 250%, 275%, 300%, 325%, 350%, 375%, 400%, 425%, 450%, 475%, 500%, 525%, and 550% of an initial dimension.

In some embodiments, the presently disclosed composition can withstand suture retention loads greater than about 50 gram-force, including about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 300%, 305%, 310%, 315%, 320%, 325%, 330%, 335%, 340%, 345%, 350%, 355%, 360%, 365%, 370%, 375%, 380%, 385%, 390%, 395%, and 400%. In particular embodiments, the composition can withstand suture retentions loads up to about 350 gram-force.

The presently disclosed modified collagen composition can comprise any one of the naturally-occurring amino acids. As used herein, the term “amino acid” includes moieties having a carboxylic acid group and an amino group. The term amino acid thus includes both natural amino acids (including proteinogenic amino acids) and non-natural amino acids. The term “natural amino acid” also includes other amino acids that can be incorporated intoproteins during translation (including pyrrolysine and selenocysteine).

Additionally, the term“natural amino acid” also includes other amino acids, which are formed during intermediary metabolism, e.g., omithine generated from arginine in the urea cycle. The natural amino acids are summarized below:

Natural Amino Acids

| Amino acid | 3 letter code | 1 letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

The natural or non-natural amino acid may be optionally substituted. In one embodiment, the amino acid is selected from proteinogenic amino acids.

Proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine. The term amino acid includes alpha amino acids and beta amino acids, such as, but not limited to, beta alanine and 2-methyl beta alanine. The term amino acid also includes certain lactam analogues of natural amino acids, such as, but not limited to, pyroglutamine. The term amino acid also includes amino acids homologues including homocitrulline, homoarginine, homoserine, homotyrosine, homoproline and homophenylalanine.

The terminal portion of the amino acid residue or peptide may be in the form of the free acid i.e., terminating in a —COOH group or may be in a masked (protected) form, such as in the form of a carboxylate ester or carboxamide. In certain embodiments, the amino acid or peptide residue terminates with an amino group. In an embodiment, the residue terminates with a carboxylic acid group —COOH or an amino group —NH₂. In another embodiment, the residue terminates with a carboxamide group. In yet another embodiment, the residue terminates with a carboxylate ester.

As disclosed hereinabove, the term “amino acid” includes compounds having a —COOH group and an —NH₂ group. A substituted amino acid includes an amino acid which has an amino group which is mono- or di-substituted. In particular embodiments, the amino group may be mono-substituted. (A proteinogenic amino acid may be substituted at another site from its amino group to form an amino acid which is a substituted proteinogenic amino acid). The term substituted amino acid thus includes N-substituted metabolites of the natural amino acids including, but not limited to, N-acetyl cysteine, N-acetyl serine, and N-acetyl threonine.

For example, the term “N-substituted amino acid” includes N-alkyl amino acids (e.g., C₁-C₆ N-alkyl amino acids, such as sarcosine, N-methyl-alanine, N-methyl-glutamic acid and N-tert-butylglycine), which can include C₁-C₆ N-substituted alkyl amino acids (e.g., N-(carboxy alkyl) amino acids (e.g., N-(carboxymethyl)amino acids) and N-methylcycloalkyl amino acids (e.g., N-methylcyclopropyl amino acids)); N,N-di-alkyl amino acids (e.g., N,N-di-C₁-C₆ alkyl amino acids (e.g., N,N-dimethyl amino acid)); N,N,N-tri-alkyl amino acids (e.g., N,N,N-tri-C₁-C₆ alkyl amino acids (e.g., N,N,N-trimethyl amino acid)); N-acyl amino acids (e.g., C₁-C₆ N-acyl amino acid); N-aryl amino acids (e.g., N-phenyl amino acids, such as N-phenylglycine); N-amidinyl amino acids (e.g., an N-amidine amino acid, i.e., an amino acid in which an amine group is replaced by a guanidino group).

The term“amino acid” also includes amino acid alkyl esters (e.g., amino acid C₁-C₆ alkyl esters); and amino acid aryl esters (e.g., amino acid phenyl esters).

For amino acids having a hydroxy group present on the side chain, the term “amino acid” also includes O-alkyl amino acids (e.g., C₁-C₆ O-alkyl amino acid ethers); O-aryl amino acids (e.g., 0-phenyl amino acid ethers); O-acyl amino acid esters; and O-carbamoyl amino acids.

For amino acids having a thiol group present on the side chain, the term “amino acid” also includes S-alkyl amino acids (e.g., C₁-C₆ S-alkyl amino acids, such as S-methyl methionine, which can include C₁-C₆ S-substituted alkyl amino acids and S-methylcycloalkyl amino acids (e.g., S-methylcyclopropyl amino acids)); S-acyl amino acids (e.g., a C₁-C₆ S-acyl amino acid); S-aryl amino acid (e.g., a S-phenyl amino acid); a sulfoxide analogue of a sulfur-containing amino acid (e.g., methionine sulfoxide) or a sulfoxide analogue of an S-alkyl amino acid (e.g., S-methyl cystein sulfoxide) or an S-aryl amino acid.

In other words, the presently disclosed subject matter also envisages derivatives of natural amino acids, such as those mentioned above which have been functionalized by simple synthetic transformations known in the art (e.g., as described in “Protective Groups in Organic Synthesis” by T W Greene and P G M Wuts, John Wiley & Sons Inc. (1999)), and references therein.

Examples of non-proteinogenic amino acids include, but are not limited to: citrulline, hydroxyproline, 4-hydroxyproline, β-hydroxyvaline, ornithine, β-amino alanine, albizziin, 4-amino-phenylalanine, biphenylalanine, 4-nitro-phenylalanine, 4-fluoro-phenylalanine, 2,3,4,5,6-pentafluoro-phenylalanine, norleucine, cyclohexylalanine, α-aminoisobutyric acid, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, pipecolic acid, 2,3-diaminoproprionic acid, tetrahydroisoquinoline-3-carboxylic acid, tert-leucine, tert-butylalanine, cyclopropylglycine, cyclohexylglycine, 4-aminopiperidine-4-carboxylic acid, diethylglycine, dipropylglycine and derivatives thereof wherein the amine nitrogen has been mono- or di-alkylated.

In one embodiment, an amino acid side chain is bound to another amino acid. In a further embodiment, side chain is bound to the amino acid via the amino acid's N-terminus, C-terminus, or side chain.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

B. Tissue-Engineering Scaffold

In some embodiments, the presently disclosed subject matter provides a tissue-engineering scaffold comprising a modified collagen or gelatin, wherein an amine of at least one or more lysine or hydroxylysine residues of the collagen or gelatin is covalently bond to a C₂ to C₁₈ substituted or unsubstituted, saturated or unsaturated aliphatic hydrocarbon chain.

In some embodiments, the scaffold supports in vitro growth of human smooth muscle cells. In particular embodiments, the human smooth muscle cells are selected from the group consisting of genitourinary cells, gastrointestinal cells, heart cells, blood vessel cells, and skin cells. In other embodiments, the presently disclosed subject matter provides an implant comprising the presently disclosed composition.

C. Method for Preparing a Modified Collagen or Modified Gelatin

In some embodiments, the presently disclosed subject matter provides a method for preparing a modified collagen or gelatin, the method comprising contacting a collagen or gelatin sample with a solution comprising a C₂-C₁₈ succinic anhydride or C₂-C₁₈ N-hydroxysuccinimide ester and a suitable reagent, such as 4-(dimethylamino)pyridine (DMAP), for a period of time to form a modified collagen.

The presently disclosed methods are suitable for use with any type of collagen including, but not limited to, fibrillar (Type I, II, III, V, XI); non-fibrillar; FACIT (Fibril Associated Collagens with Interrupted Triple Helices) (Type IX, XII, XIV, XIX, XXI); short chain (Type VIII, X); basement membrane (Type IV); multiplexin (Multiple Triple Helix domains with Interruptions) (Type XV, XVIII); MACIT (Membrane Associated Collagens with Interrupted Triple Helices) (Type XIII, XVII); and other (Type VI, VII). The most common types of collagen include: Type I skin, tendon, vasculature, organs, bone (main component of the organic part of bone); Type II: cartilage (main collagenous component of cartilage); Type III: reticulate (main component of reticular fibers), commonly found alongside type I; Type IV: forms basal lamina, the epithelium-secreted layer of the basement membrane; and Type V: cell surfaces, hair, and placenta. In certain embodiments, the collagen is selected from the group consisting of collagen I, collagen II, collagen III, and collagen X. In particular embodiments, the collagen is collagen I. In some embodiments, the collagen is a vitrified collagen. In other embodiments, the collagen is not a vitrified collagen. In particular embodiments, the method further comprises neutralizing the collagen. In other embodiments, the collagen is a gelatin.

Further, one of ordinary skill in the art would recognize that other anhydrides could be suitable for use with the presently disclosed methods. An organic acid anhydride has the following general structure:

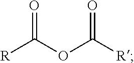

wherein R and R′ are each alkyl chains, including C₁-C₂₀ alkyl chains. In particular embodiments, the anhydride can be a cyclic anhydride, of which succinic anhydride is an example:

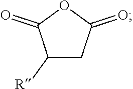

which can be further substituted with a C₂-C₁₈ aliphatic hydrocarbon chain (R″), wherein the C₂-C₁₈ aliphatic hydrocarbon chain can be linear or branched and, in some embodiments, can include one or more carbon-carbon double bonds. Other cyclic anhydrides, such as substituted maleic anhydrides, substituted maleimides, substituted succinimides, and the like are suitable for use with the presently disclosed methods.

Further, one of ordinary skill in the art would recognize that other nucleophilic catalysts in addition to DMAP are suitable for use in the presently disclosed methods, including pyridine, N,N′dicyclohexylcarbodiimide (DCC), imidazole, 1,8-diazabicyclo[5.4.0]undec-7-ene (DBU), dimethylformamide (DMF), and hydroxybenzotrizole (HOBt), or any suitable tertiary amine.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an alkyl group substituent may have another substituent off it, such as another alkyl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.” More particularly, the term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, +100% in some embodiments ±50%, in some embodiments 20%, in some embodiments 10%, in some embodiments 5%, in some embodiments 1%, in some embodiments 0.5%, and in some embodiments 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Materials

Bovine skin Collagen type I was purchased from CosmoBio (Tokyo, Japan). Dodecenylsuccinic anhydride (DDSA or 2-Dodecen-1-yl succinic anhydride, M_{w} 266.38 Da), 4-(dimethylamino)pyridine (DMAP) (M_{w} 122.17 Da), and N-(3-dimethylaminopropyl)-N′-ethylcarbodiimide hydrochloride (EDC, M_{w} 191.71 Da) were purchased from Sigma-Aldrich (St. Louis, Mo.). Dimethyl Sulfoxide (DMSO, anhydrous, 99.8+%) was purchased from Alfa Aesar (Ward Hill, Mass.), and N-hydroxysuccinimide (NHS, M_{w} 115.09 Da) was purchased from Thermo Fisher Scientific (Waltham, Mass.). All other reagents are purchased from Sigma-Aldrich unless otherwise mentioned.

### Example 2

### Engineering Collagen Scaffolds

Collagen scaffolds were prepared by neutralizing type I collagen (5 mg/mL in HCl, 10 ml) with 8.8 mL of Dulbecco's Modified Eagle's Medium (DMEM) with 1 g/L D-Glucose, L-glutamate, 110 mg/L Sodium Pyruvate (Life Technologies, NY), 1.0 mL of fetal bovine serum (FBS) and 0.2 mL of HEPES (×1, 1M) solution at 4° C.

2.1 Collagen discs: The setup for the collagen disc was made with two customized molds in a hollow and cylindrical-shape balloon chamber. Each mold had a porous 90-micron, hydrophilic, ⅛″ thick polypropylene (SCI Inc., Arizona) custom-cut disc, which was snap-fitted into the mold. A round bottom Erlenmeyer flask was used in a double trap set up connected to the vacuum. The neutralized collagen solution was allowed to undergo fibrogenesis for one hour followed by applying vacuum to remove excess fluid while bringing the top mold down when the curvature of the balloon was slightly in an hourglass shape due to fluid removal.

2.2 Collagen tubes and hemispheres: The freshly mixed neutralized collagen solution was injected into a balloon chamber with a sintered plastic mold (RKI Instruments rod-JJS Tech., IL) with either tubular or hollow shape. After about 1 h of incubation at 37° C. for fibrogenesis, collagen was further condensed by extracting water either under partial vacuum or against the contractile pressure of the balloon. The balloon chamber was opened to remove the tubular scaffold. In some cases, the tube was vertically kept on a rotating plate in a humidity-chamber (39° C., 40% RH) for 20 h (known as the dehydrothermal treatment) for further drying the scaffold. On rehydration, the tube was slowly pulled off the mold.

### Example 3

### Material Synthesis and Composition

3.1 Collagen Modification: DDSA was dissolved in anhydrous DMSO to make a homogenous solution (different concentrations, 2 mg/mL to 20 mg/mL). To this solution, DMAP was added to make a concentration of 2.0 mg/mL. Collagen type I scaffold (either vitrified or not vitrified) was soaked in PBS (pH 7.4) solution for 15 minutes, dab-dried, and submerged in the DDSA/DMAP solution and left on a shaker at room temperature for different time periods (typically, 5 min to 1 h). After the reaction, modified collagen was taken out and washed three times with PBS (5 min each wash). In a similar procedure, other collagen samples were modified with reactants having varying carbon chain lengths.

3.2 Crosslinking of Collagen Scaffolds: Scaffolds were crosslinked at room temperature with either EDC/NHS (EDC 1.0 mg/mL, NHS 2.0 mg/mL) or transglutaminase (Sigma-Aldrich, 1.5 units/mg, guinea pig liver lyophilized powder). EDC and NHS were added to DMSO solution with DDSA and DMAP and allowed to react for 20 minutes at room temperature while shaking. For gamma-radiation based crosslinking, collagen was kept submerged in PBS and exposed to LINAC gamma radiation for a total dose of 13.4 kGy at 18 Gy/min for 12.5 h. After the reaction, the scaffold was taken out and washed three times with PBS (5 min each wash).

3.3 Determination of Amine Content: To determine free amine groups in collagen scaffolds, an assay of 2,4,6-trinitrobenzene sulfonic acid (TNBS) was performed. In brief, a small portion of dried collagen scaffolds were pre-weighed and added with 0.5 mL of 4% (w/v) NaHCO₃ solution and 0.5 mL of a freshly prepared solution of 0.05% (w/v) TNBS. After reacting it for 2 h at 40° C., 1.5 mL of 6 M HCl was added and left for 90 min at 60° C. for hydrolysis. The solution was diluted with 2.5 mL of distilled water, brought to room temperature, and measured for its absorbance at 352 nm using a Fluostar Optima spectrophotometer. Controls (blank samples) were prepared using the same procedure, except that hydrochloride acid was added prior to addition of TNBS solution. The absorbance value for the blank sample value was subtracted from that of samples. These absorbance values were correlated to the concentration of free amine groups using a calibration curve obtained with glycine in an aq. NaHCO₃ solution. Three replicates were used for each condition.

### Example 4

### Material Characterization

4.1 Nuclear Magnetic Resonance (NMR) and Fourier-Transform Infrared (FTIR) Spectrometries: The modified collagen was characterized by ¹H-NMR in D20 and DMSO-D6, respectively using a Bruker Advance III 500 MHz NMR spectrometer, and by FTIR-ATR using the Thermo-Nicolet Nexus 670 FTIR Spectrometer with the Nicolet Smart Golden Gate and the KRS-5 ATR accessory. The Bruker TopSpin software was used to process the ¹H-NMR spectrum.

4.2 Differential Scanning Calorimetry (DSC): DSC was performed using a DSC 8000 (Perkin Elmer, Waltham, Mass.) to determine the melting temperature of collagen scaffolds. Pre-weighed hydrated samples were prepared and placed in aluminum sample pans and crimp-sealed with aluminum lids supplied by the manufacturer. Temperature ramp of 5° C./min over a range of 20° C. to 70° C. was performed. DSC thermograms were analyzed using Pyris Series Thermal Analysis software (Perkin Elmer) version 10.1.

4.3 Circular Dichroism (CD). CD spectra of collagen solution at 0.125 mg/mL at pH 3.0 (hydrochloride solution), over the range of 190 nm to 250 nm were obtained using Aviv Biomedical spectrometer. Circular dichroism was recorded at 222 nm (absorption bands of the collagen triple helix) for different solutions at room temperature.

4.4 Transmission Electron Microscopy (TEM). Samples were fixed in 3% paraformaldehyde, 1.5% glutaraldehyde, 5 mM, 5 mM CaCl₂, 2.5% sucrose, and 0.1% tannic acid in 0.1 M sodium cacodylate buffer, pH 7.2. After buffer rinse, samples were post fixed in 1% osmium tetroxide for 1 h on ice in the dark. Following a rinse with distilled water, the samples were stained with 2% aqueous uranyl acetate (0.22 μm filtered) for 1 h in the dark, dehydrated using a graded series of ethanol, and embedded in Eponate 12 resin (Ted Pella, Redding, Calif.). Samples were polymerized for 2-3 days at 37° C. and were stored at 60° C. overnight. Thin sections, 60-90 nm, were cut at a depth of 50 m from the surface with a diamond knife using a Reichert-Jung Ultracut E ultramicrotome and placed on naked copper grids, stained with 2% uranyl acetate in 50% methanol, and observed using a Philips/FEI BioTwin CM120 TEM and the images were processed with Image-J software (US National Institutes of Health, Bethesda, Md.).

4.5 Two dimensional-X-ray diffraction study. The powder XRD system (X-pert Pro. Phillips. Cu anode, Kalpha 1.54184 Angstrom) was used to determine the composition of powder and film samples. Samples are mounted flat to slides or to zero background holders that provide minimal scattering for analysis. Often the samples were either continuous flat-exposed or were crushed into fine powder for analysis.

### Example 5

### Temperature, pH and Enzymatic Stability

5.1 Swelling ratio: Samples were soaked in PBS (pH 7.4) and kept at room temperature (25° C.), and the wet weights were measured at time t=0, 0.5, 1, 2, 3, 4, 6, 13.5, and 24 hours. The swelling ratio of each sample was plotted over time: Swelling ratio=(Wet weight of the sample-Dry weight)/Dry weight of the sample.

5.2 Temperature stability test: Scaffolds were evaluated for their stability in PBS (pH 7.4) at three temperatures (4° C., 25° C., and 37° C.). After measuring it initially, the wet weight was measured again after 24 hours. The swelling ratio of each sample was plotted against the varying temperatures.

5.3 pH stability test: Samples were soaked in aqueous solutions with varying pH (4.0, 5.0, 6.0, 7.4 and 8.5) at room temperature (approximately 25° C.). The wet weights were measured at time t=0 and 24 h. The swelling ratio was plotted against the varying pH solutions.

5.4 Enzyme degradation test: The degradation of scaffolds was examined in the presence of two enzymes: Collagenase IV and Trypsin. Collagenase IV was dissolved in PBS (pH 7.4) at a concentration of 1.0 mg/mL, and a concentration of 0.25% for Trypsin-EDTA was used. The dry weights of samples were measured at time t=0 h followed by soaking them at 37° C. in either collagenase IV or Trypsin solutions. The wet weights of samples were measured at time t=0 and 24 h. After 8 days, partial undegraded samples remained; therefore, a second dose of enzyme was added to the solution. The wet weights of samples were measured at time t=24 h after the second dose, and at t=9 days after the first dose. The swelling ratio for each sample was plotted against each enzyme.

### Example 6

### Flow and Mechanical Properties of the Scaffold

6.1 Rheological properties: To examine the viscoelastic properties of the hydrated scaffolds, a strain sweep and temperature ramp scan were performed with the ARES-G2 rheometer (8-mm diameter, parallel plate geometry, stainless steel, serrated). Temperature ramp was performed from 25° C. to 70° C., at a ramp rate of 5° C./min, with an angular frequency of 10 rad/s, and a strain percent at 1% (within linear viscoelastic region, determined by strain sweep). During the strain sweep test, the parameters were set to: temperature of 25° C., frequency of 10 Hz, and strain percentage from 0.1% to 100%. The storage modulus (G′), loss modulus (G″), tan (delta), and complex viscosity were plotted against temperature.

6.2 Mechanical Testing: Rectangular samples were tested with a 5N load cell in the MTS Criterion™ Model 43. For suture tests, samples were threaded with a suture string that was knotted to hold the sample during the experiment. For tensile tests, samples were put in a paper-based custom apparatus to hold them during the experiment. The same setup was used for hysteresis tests. For hysteresis experiments, samples were tested for three cycles of stretching and restoring. All data were recorded and analyzed in Microsoft Excel and GraphPad Prism.

### Example 7

### In Vitro Biocompatibility

7.1 Pretreatment of scaffolds for cell culture: Scaffolds were treated with 0.2% peracetic acid and 4% ethanol (in PBS, pH 7.4) for 6 h followed by three PBS washes for at least one hour each prior to cell-seeding.

7.2 Cell culture and expansion: Human Urothelial Cells (hUCs) and human SMCs (hSMCs) were purchased from Sciencell research laboratories (Carlsbad, Calif.). Experiments were performed with all cell types between passage 3 and 4. SMCs were cultured in SMC medium consisting of a basal SMC medium (Sciencell, CA) with SMC growth supplement, 10% FBS and 1% Penn-Strep. UCs were cultured on Poly (L-Lysine) (Sciencell, CA) coated cell culture flasks till P3 in a growth medium that consists of basal UC medium (Sciencell, CA) with UC growth supplement and 1% Penn-Strep (Life Technologies). Medium was changed every 2-3 days for all cell types.

7.3 Cell Seeding on collagen scaffolds: SMCs seeded were allowed to attach to small pieces of (5 mm×5 mm) scaffolds in cell culture plates and kept in SMC growth medium at 37° C. in a cell culture incubator. One million cells were seeded in each side of the scaffold in an interval of 30 min. hSMCs growth medium was changed every 2-3 days till the completion of the experiment.

7.4 Cell morphology seeded on scaffolds by SEM: Scaffolds were fixed overnight at 4° C. in 2.5% glutaraldehyde in 100 mM sodium cacodylate buffer and 0.1% tannic acid (pH 7.2-7.4). Samples were washed in a 100 mM sodium cacodylate buffer with 3% sucrose and 3.0 mM MgCl₂. Samples were post-fixed in 0.8% potassium ferrocyanide and reduced with 1% osmium tetroxide for one hour on ice, in the dark, followed by H₂O washes. Samples were dehydrated using a graded ethanol series before hexamethyldisilanaze (HMDS) rinses. After drying overnight in a desiccator, dry samples were mounted to Pelco SEM stubs using carbon tape and sputter coated with 10 nm of gold palladium and imaged on a LEO 1530 FESEM.

### Example 8

### In Vivo Biocompatibility

8.1 Subcutaneous biocompatibility study: Animal studies were performed with protocols that were approved by the Johns Hopkins University Hospital Animal Care and Use Committee. In brief, four black male mice (C57BL/6 strain) were anaesthetized with isoflurane and two samples (4×4 mm size) of control collagen, modified collagen graft (also crosslinked with EDC/NHS) were grafted in anterior ventral side of mice. Two pieces from each group were implanted into each mouse for a total of four pieces for each group. After one week had passed, the mice were sacrificed and the grafts were harvested out for histological analysis.

8.2 In vivo biocompatibility in a bladder augmentation model. Sprague Dawley white male rats were anaesthetized with isoflurane. Rats were divided into three different groups: cystectomy control (group 1), pericardium group (group 2, Tutoplast from Coloplast™), and modified pericardium (group 3). For group 1, sterile technique was used to perform a cystectomy surgery on rat bladder with a resulting incision of 4 mm. For groups 2 and 3, pericardium grafts were first imaged under a 20× microscope and then, using sterile conditions, implanted into the rat after an incision had been performed on the bladder. The grafts were harvested after 28 days and analyzed for their ability to allow growth of urothelium and muscle layers by histology and RT-qPCR.

### Example 9

### Histology

Morphological assessment of cell-seeded scaffolds was performed with a procedure published earlier by Atala et al., 2006. Histology was performed on formalin fixed, paraffin embedded tissues, which were sectioned at 6 microns onto glass micro-slides. Finally, sections were counterstained with hematoxylin and eosin (Richard-Allen Scientific, Kalamazoo, Mich.), and Trichrome staining in accordance to a typical histology protocol. Samples were imaged with Zeiss Discovery V2 dissection imaging microscope.

### Example 10

### Statistical Analyses

Unpaired t-test with Welch's correction was performed to determine any statistical significance in mean values unless stated otherwise (n=3). Statistical significant values with p<0.05 were marked as*.

### Example 11

### Results

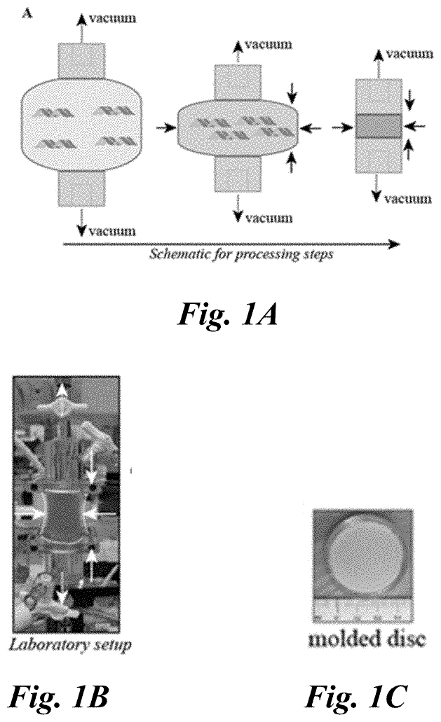

11.1 Fabrication of collagen scaffold: In a custom-made balloon chamber (FIG. 1A, FIG. 1B, and FIG. 1C), collagen I solubilized in acidic solution was neutralized to undergo fibrogenesis at room temperature for 1 h. The extra solution was removed by applying vacuum from both sides along with bringing the upper and lower molds closer, periodically. When almost all the solution was removed, a disc shaped-collagen scaffold was obtained. The extra liquid was further removed by gently drying with a paper towel. The disc further underwent treatment for vitrification or chemical crosslinking as needed.

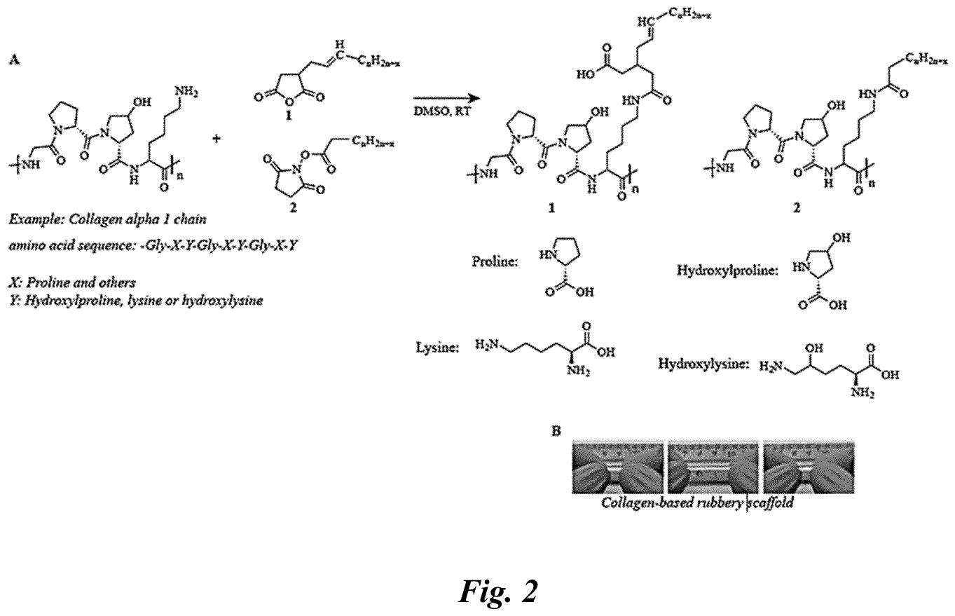

11.2 Synthesis and composition of elastomeric collagen: Amines that are present in collagen via lysine or hydroxylysine were modified with aliphatic chains of varying length (C₂-C₁₈, FIG. 2A). Due to the poor solubility of the investigated aliphatic molecules in water, anhydrous DMSO was used to react them with collagen. Pre-fabricated collagen scaffold was soaked in PBS (PH 7.4) followed by an addition of aliphatic molecules in DMSO at ambient temperature. Modified collagen with C₁₂ chain length in DMSO when washed with PBS became highly compliant and showed rubbery characteristics. Surprisingly, molecules with C₉-C₁₂ chain length yielded compliant materials; however, other aliphatic molecules either did not change the gross properties or made the resultant material brittle.

To eliminate the possibility of the reaction being specific to anhydrides containing aliphatic chains, NHS was reacted with a substituted C₁₂ aliphatic chain, which also yielded compliant materials. This observation implies that aliphatic chain length size-C₉-C₁₂ is unique and may possibly be independent of the reactive functional groups (N-hydroxysuccinimide ester (NHS) vs succinic anhydride). To investigate if the same property can be achieved, collagen was reacted with various molecules having different chain lengths and composition (see Table 1). Replacing C₁₂ with 4×(C—C—O) yielded a swollen gel that was fragile. Molecules with less than C₉ length and greater than C₁₂ did not yield a rubbery material.

TABLE 1
Modified collagen with different groups
and their gross visual properties

| n | x | Yes/No | name | observation |
|---|---|---|---|---|
| 9 | 1 | No | azidoacetic acid NHS ester | No change |
| 9 | 1 | No | propargyl acid NHS ester | No change |
| 9 | 1 | No | Caprylic acid NHS ester | No change |
| 9 | 1 | Yes | Lauric acid NHS ester | Rubbery |
| 6 | 1 | No | Myristic acid NHS ester | Fragile |
| 6 | 1 | No | mPEG4-NHS ester | Swollen, gel |
| 9 | 1 | Yes | (2-Nonen-1-yl) succinic anhydride | Rubbery |
| 9 | 1 | Yes | (2-Dodecen-1-yl) succinic anhydride | Rubbery |
| 15 | 1 | No | (2-Octadecen-1-yl) succinic anhydride | Fragile |
| 8 | F | No | Perfluorodecanoic acid NHS ester | Fragile |

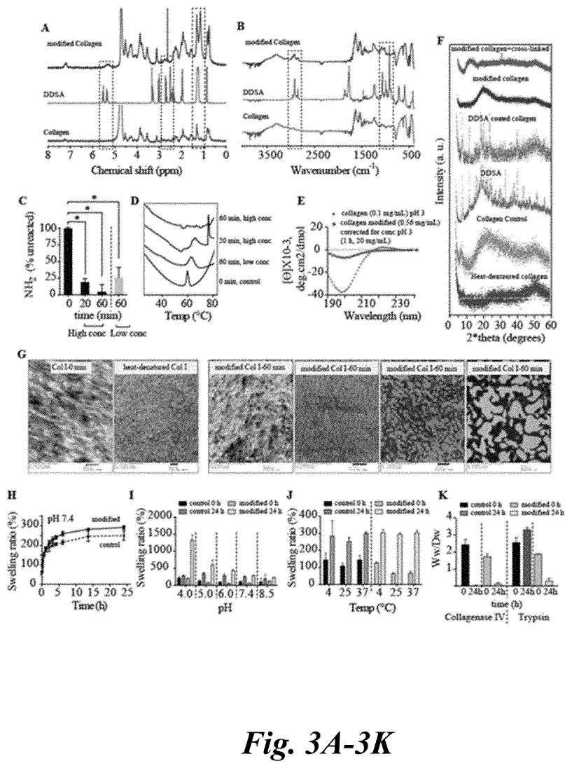

1.3 Characterization of the resultant modified collagen. Conjugation of the C₁₂ aliphatic molecule to collagen was confirmed by ¹H-NMR (FIG. 3A) and FTR-ATR (FIG. 3B). ¹H-NMR resonances corresponding to an unsaturated double bond in DDSA at approximately 5.5 ppm was evident in the resultant material. Similarly, FTIR-ATR peaks at about 850 cm⁻¹ (DDSA) and 2750 cm-1 (DDSA) also confirmed the C₁₂ modification of collagen. TNBS assay (FIG. 3C) further confirmed the consumption of amines over time on reaction with DDSA (20 mg/mL-high concentration, 2 mg/mL-low concentration). Within 20 min of reaction, approximately 80% amines were reacted with DDSA, while after 1 h of reaction approximately 90% amines were consumed. At a lower concentration of DDSA, approximately 25% amine was unreacted after 1 h. DSC thermogram (FIG. 3D) demonstrated that collagen melting peak (wet in PBS pH 7.4, about 60-61° C.) on reaction with DDSA overtime gets broader and eventually disappears after 60 min as seen at the higher concentration of DDSA. While, a broad melting peak remains after 60 min of the reaction at the lower concentration of DDSA. The broader and poorly defined melting peak implies that the resultant material partially lost its triple helix configuration. At a higher concentration of DDSA and a longer time of reaction when approximately all amines are consumed, there is no well-defined melting peak indicating possible unfolded and loss of triple helix configuration.

The resultant material was further investigated by circular dichroism (FIG. 3E) spectroscopy. The typical collagen-I triple helix signal in circular dichroism has a weak positive band at the wavelength of 222 nm and a strong negative band at the wavelength of 200 nm. A reduced negative signal and lack of a positive band at 222 nm imply that the material resembles features of denatured collagen or unfolded triple helix in collagen. A two-dimensional XRD spectroscopy (FIG. 3F) further showed that the modified collagen has two major peaks at about 20 degrees and about 50 degrees similar to the control collagen, however, a peak at 5-6 degrees is not present. However, an EDC/NHS crosslinked modified collagen sample exhibited peak corresponding to triple helix with disappearance of the broad amorphous peak at about 20 degrees and appearance of a sharp peak at about 35 degrees, which also is found in DDSA.

It is possible that on simultaneous crosslinking with EDC/NHS and aliphatic modification, there are regions of triple helix in molecular networks, as well as some unfolded portion of the chain with aliphatic modifications. The resultant EDC/NHS crosslinked and aliphatic modified collagen sample was strong, flexible and suturable. It is thought that it may be possible to balance the compliance with strength and triple helix content in modified collagen. While, DSC, circular dichroism and XRD suggest a presence of unfolded and lack of triple helix configuration, TEM performed on these samples did not rule out the complete absence of collagen type-I fibril band from the material (FIG. 3G). The modified collagen reacted for the higher concentration of DDSA for longer time (with almost all free amines consumed, FIG. 3C and with no well-defined characteristic melting peak by DSC) was uniquely structured in addition to presence of some fibril band structure. TEM images (FIG. 3G, white arrows show typical band structure of collagen I) showed that the resultant material has a phase-separated morphology (dark and light staining). These results imply that possibly on reaction with DDSA, collagen triple helix adopts an unfolded molecular structure and phase separates in hydrophobic and hydrophilic domains due to aliphatic DDSA units and random coil configuration of unfolded collagen chains. However, it is thought that more in depth mechanistic and morphological study is needed to characterize the resultant material. It is highly probable that the resultant material is combination of triple helix and unfolded collagen type I with aliphatic chain modifications. It also may be possible that the material from deep inside (with reference to the outer surface) might have preserved triple helical structure while the top layers, which are exposed to the highly concentrated DMSO-DDSA solution are unfolded.

The modified collagen scaffold was further characterized for its swelling ability in different buffer solutions (with pH values of 4.0, 5.0, 7.4 and 8.4, FIG. 3H for pH 7.4, and FIG. 3I) and investigated its performance at different temperatures (4° C., 25° C. and 37° C., FIG. 3J) and in enzymatic solutions of Collagenase and Trypsin (FIG. 3K). The equilibrium-swelling ratio at room temperature in PBS (pH 7.4) for modified collagen is approximately 290% vs. 250% of control. An observation of higher swelling ratio despite it being modified with hydrophobic units indicates that the modified material might also have an unfolded random coil structure, which is relatively less compact than the rod like structures of collagen fibers. Although the modified collagen swelled multiple folds in acidic pH, the difference in swelling compared to collagen-control decreased with increasing pH at the constant temperature (FIG. 3I). Similar to collagen control, there was no significant difference observed in swelling ratio from 4° C. to 37° C. (FIG. 3J). The modified collagen was approximately fully digested in collagenase similar to control collagen; however, some undegraded material was found in trypsin solution. It is known that Trypsin can digest unfolded collagen chains; therefore, this finding implies that it is possible to that the modified collagen has some intact triple helix configuration.

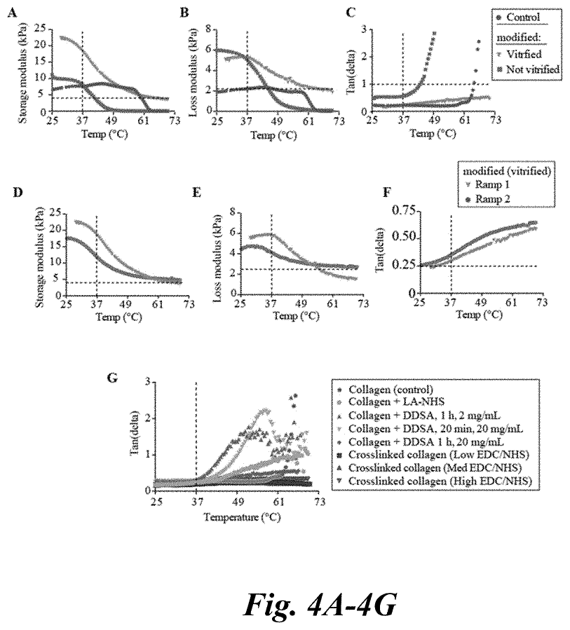

11.4 Rheological (flow) properties of modified collagen: Collagen control whether vitrified (also known as a dehydrothermal crosslinking process) or not exhibited a relatively constant storage, loss moduli and tan(delta) (loss modulus/storage modulus) values that drastically change around its melting temperature (data shown only for unvitrified collagen sample, about 60-61° C., the mid point of the moduli drop in FIG. 4A and FIG. 4B). While it is obvious that collagen fibrils being an organized-stacked 3D network structure lacks an energy dissipation mechanism (viscous component) and undergoes a mechanical failure mostly by the brittle fracture, a compositional modification of collagen with hydrophobic units should enhance the viscous component of the material (FIG. 4B, loss modulus). Vitrified collagen on modification exhibits higher storage modulus values throughout the temperature range of measurement compared to the unvitrified modified collagen samples. It is possible that vitrification, introduces some crosslinking in vitrified collagen fibers that in turn enhances the storage modulus in modified collagen sample. While the storage modulus remains higher for vitrified samples compared to the unvitrified sample, the loss modulus values remain similar until 37° C. Both the storage and loss moduli values for samples that are modified from unvitrified collagen reduce to minimal and the material undergoes a liquid flow at higher temperatures. The samples that are modified from vitrified collagen tend to exhibit a plateau around collagen melting point after a relatively slower drop in the values after 37° C.

An important parameter, Tan (delta), is an indicator of material solid-gel state. A plot of tan delta vs. temperature indicates the flow behavior of the material (liquid tan delta greater than one, solid tan delta less than one). Collagen control samples exhibit an approximately constant Tan (delta), which changes at its melting point. For modified (unvitrified collagen) samples, the transition happens at a quite lower temperature just next to 37° C. (FIG. 4C). Tan delta values for modified (vitrified collagen) samples start linearly going up after 37° C. Since the storage, loss moduli were tending to plateau and tan delta being less than 1.0, the modified samples (from vitrified collagen) hold its geometry and structurally remained unchanged. When the sample was allowed to cool and underwent another round of temperature scan, the values were comparable to the 1^{st} ramp that supports our observation (FIG. 4D, FIG. 4E, and FIG. 4F). The flow behavior of the materials that were synthesized were further investigated with different reagents. The collagen samples also were crosslinked while modifying with aliphatic chains, which showed that increasing the concentration of EDC/NHS (henceforth increasing crosslinking) decreases the tan (delta) values. While, low and medium concentrations of EDC/NHS with aliphatic chains (fixed) remained compliant, the higher concentration of EDC/NHS decreased the compliance of the scaffold (data not shown). Similar to DDSA modification, LA-NHS modification also had similar tan (delta) profiles that of DDSA modified sample (FIG. 4G). In summary, the material investigated remains in solid state and with enhanced viscous components at 37° C., which may help the scaffold to be pliant, flexible, and tougher and not undergo brittle fracture.

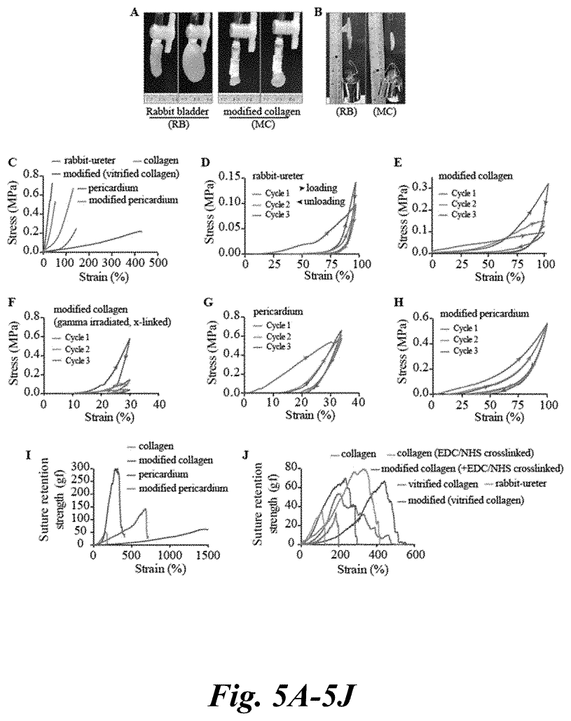

11.5 Biomechanical properties of elastomeric collagen: To investigate if the modified collagen possessed sufficient strength, compliance, toughness and suture retention strength (FIG. 5A-FIG. 5J), mechanical testings were performed on specimens. The tensile stress-strain curves for modified collagen were characterized by a slope-changing curve (FIG. 5C). As shown in FIG. 5C, the modified collagen can elongate to more than 400% of initial length compared to only 35% of collagen-control, and approximately 120% of the rabbit ureter (longitudinal direction), while the modified pericardium elongated up to 120% compared to 30% of pericardium control. While, samples' ability to elongate enhanced, the ultimate tensile strength of the samples decreased from 0.5 MPa of collagen-control to about 0.21 MPa, a value similar to that of a rabbit ureter. For modified pericardium tissue, it dropped from 0.65 MPa to 0.60 MPa. However, the area under the stress-strain curve, also known as the toughness or its ability to absorb energy (area under the curve in stress-strain graph), increased significantly. The modified collagen was 2.7 times tougher than collagen-control, 4.0 times tougher than the rabbit ureter. The modified pericardium was 1.75 times tougher than the control-pericardium. The modified material's ability to undergo repetitive stretching was further evaluated as represented by the tensile-hysteresis curve (in three cycles of experiments, FIGS. 5D-FIG. 5H). Rabbit ureter and aliphatic-modified samples were investigated up to 100% strain while collagen and pericardium were investigated up to 30% strain (below the breaking strain). Tensile hysteresis curve is an important indicator of the energy loss during the repetitive deformation and recovery phases while loading und unloading. Soft tissues, including urinary bladder exhibit viscoelastic behavior to some extent as some energy is dissipated during filling and voiding. All investigated samples showed hysteresis loops within the range of the strain range investigated. Three cycles of loading and unloading were performed. The biological tissues, both rabbit ureter and pericardium, showed relatively less hysteresis energy loss compared to the modified collagen (2^{nd} and 3^{rd} cycles). The experiments were performed in a closed chamber, however, without samples being continuously merged in PBS, which caused some samples to undergo overtime dehydration (except the modified pericardium, in all samples, the peak stress in 3^{rd} cycle is higher compared to 1^{st} and 2^{nd} cycles). The area calculated in between loading and unloading represents hysteresis energy loss due to internal molecular friction and represent viscoelastic nature of the material. The area of the stress-strain curve was calculated for the 2^{nd} cycle as a representative of dissipated energy among different samples. For the rabbit ureter (within 90% strain), control-pericardium (within 30% strain) and modified pericardium (within 100% strain), the loss in energy was about 37% of the total energy or toughness (area of the stress-strain curve for the 2^{nd} loading cycle), while for gamma-irradiated collagen-control (within 30% strain) and the modified collagen (within 100% strain), the loss in energy was 66% of the total energy. It had been observed earlier that the area of the hysteresis loop does not change after a few cycles of loading and unloading (9 to 10 cycles) until the curves overlap. Therefore, earlier cycles are considered as preconditioning steps; however, in these experiments, the number of cycles was limited to 3 to avoid drying of samples in lack of continuous exposure to PBS and considered 2^{nd} cycle to compare the values among different groups.

The suture retention strength values of the modified samples were further evaluated (FIG. 5I-FIG. 5J). The suture retention strength values for collagen control and modified collagen were comparable (approximately collagen control 50 gm force vs. modified collagen approximately 60 gm force), while for modified pericardium it decreased from 300 gm force to 140 gm force; however, the elongation at break increased multiple folds due to modified materials' ability to stretch under the applied load (modified collagen 1500% vs 100% of collagen control; modified pericardium ˜750% vs 250% of pericardium control, FIG. 5I). This result indicates that while the implant sutured with the neighboring tissues, it will be able to undergo stretching without failing due to rupture against the fluid pressure. The suture retention strength values and profiles for modified collagen were similar to the rabbit ureter. The suture retention strength values for collagen control increased on crosslinking by either EDC/NHS or vitrification however with a decease in elongation at break (FIG. 5J). While, the modified collagen showed higher elongation values at break points.

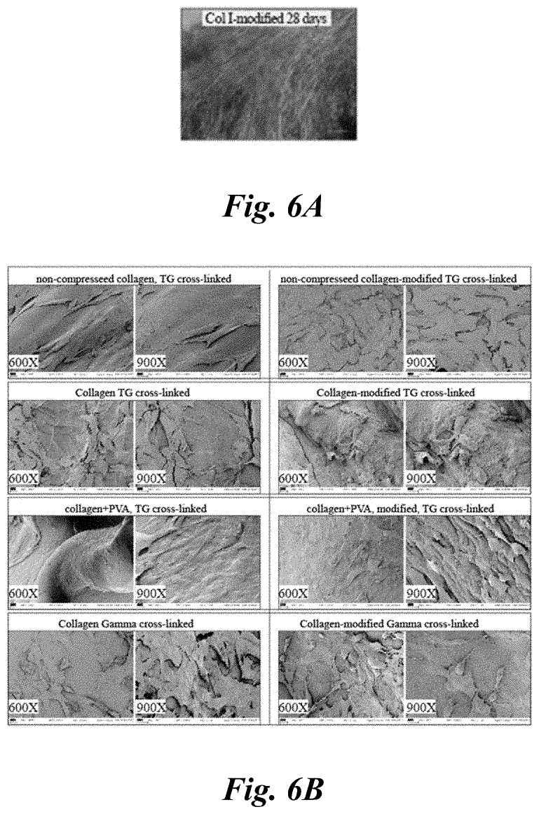

11.6 In vitro cell culture on modified collagen scaffold: The modified collagen supported growth of human smooth muscle cells. Aligned and oriented hSMCs were observed (FIG. 6A) after 28 days of cell culture in a static cell culture condition. Transglutaminase (TG) crosslinked non-condensed collagen gels showed expanded cell morphology compared to an elongated morphology on unmodified TG crosslinked collagen sample after 3 days of culture; however, there was no apparent difference in cell morphology among modified and unmodified condensed collagen samples (more expanded, FIG. 6B). hSMCs cultured for 3 days on crosslinked and modified collagen samples (transglutaminase crosslinked with or without added Poly(vinyl alcohol) or PVA particles or gamma irradiated crosslinked) grew well in all the conditions with similar morphology to control crosslinked compressed collagen samples (FIG. 6B). SEM images of cell morphology are indicative of SMCs favoring a more expanded morphology in condensed collagen samples with or without modification.

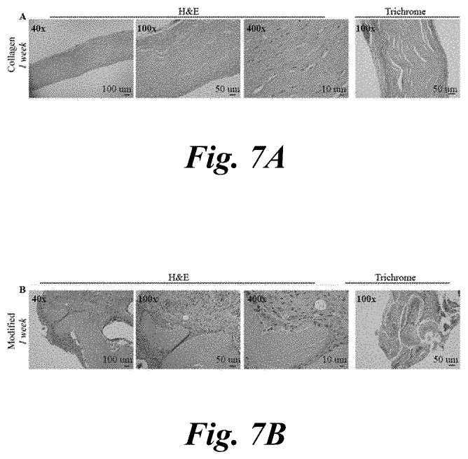

11.7 Subcutaneous biocompatibility: All samples were present within 1^{st} week of subcutaneous implantation. Condensed collagen allowed cells to penetrate along the layers although sparsely by 1 week (FIG. 7A-FIG. 7B, H&E and Trichrome staining). In contrast, modified collagen sample was observed to be mostly inert at 1 week; however, cells were observed at the interface of the graft with the surrounding tissues. Substantial inflammation at the graft site due to these materials was not observed.

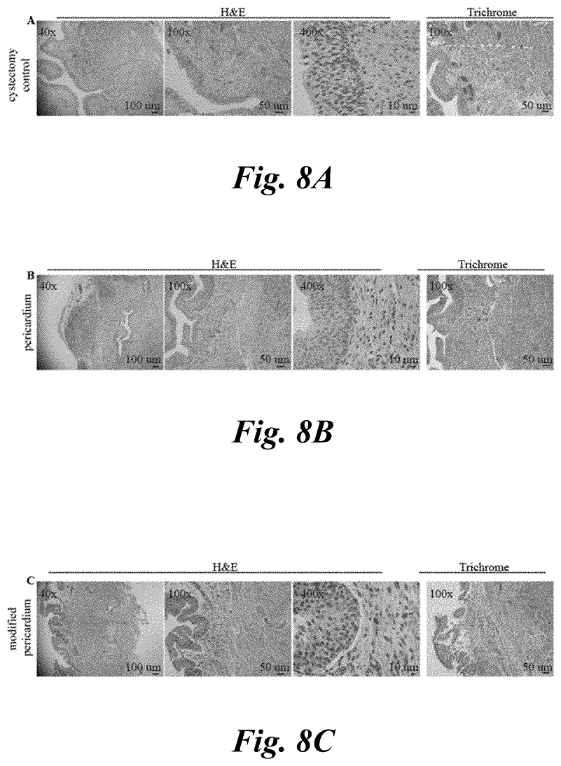

11.8 Bladder augmentation: The biocompatibility of a commercially available decellularized tissue (Tutoplast, pericardium tissue) and its DDSA modified version into a bladder augmentation model were further evaluated. All samples were found to support urothelium regeneration (FIG. 8A-FIG. 8C). Histologically, cells were able to penetrate throughout the scaffold. A discontinuity of urothelium to the bulk of the graft for the modified sample was observed, however, which may have happened either due to intrinsic difference in regeneration of urothelium attachment to the bulk or a processing artifact (FIG. 8A-FIG. 8C). Since a discontinuity at the interface of the graft to the host tissue also was observed, it is possible that altered chemical composition of the material causing reduced adhesion at the interface.

In summary, a collagen-based formulation was developed that can be used to fabricate scaffolds that are rubbery in nature, strong, yet compliant, have sufficient suture retention strength, and are biocompatible. The scaffolds made from this material will be very useful to create/regenerate soft tissues.

### Example 12

### Elastin-Free Gelatin-Based Material Composition for Developing Elastomeric Materials

12.1 Materials. Gelatin type A (Porcine, bloom 300), dodecenylsuccinic anhydride (DDSA or 2-Dodecen-1-yl succinic anhydride, M_{w} 266.38 Da), 4-(Dimethylamino)pyridine (DMAP) (M_{w} 122.17 Da), and N-(3-Dimethylaminopropyl)-N′-ethylcarbodiimide hydrochloride (EDC, M_{w} 191.71 Da) were purchased from Sigma-Aldrich (St. Louis, Mo.). Dimethyl Sulfoxide (DMSO, anhydrous, 99.8+%) was purchased from Alfa Aesar (Ward Hill, Mass.), and N-Hydroxysuccinimide (NHS, M_{w} 115.09 Da) was purchased from Thermo Fisher Scientific (Waltham, Mass.). All other reagents are purchased from Sigma-Aldrich or Johns Hopkins Core supply facilities unless otherwise mentioned.

12.2 Material Synthesis and Composition. Gelatin was dissolved in 60% ethanol (with distilled water) to make a homogenous solution of 100 mg/mL at 65° C. DDSA (250 mg dissolved in 1.0 mL of DMSO) was added to this solution followed by an addition of DMAP (as a nucleophilic reagent, 25 mg in 1 mL of DMSO). The mixture was allowed to react for an hour while shaking at 200 rpm. The hot solution was poured into a rectangular Teflon mold (3 cm×2 cm×2 mm) and allowed to gel at room temperature. The casted gel when cold was washed with water several times.

12.3 Results and Discussion. Gelatin, a derivative of collagen, is one of the widely available and relatively inexpensive natural biomaterials, which is utilized for a wide range of medical applications, including in pharmaceutical engineering, drug delivery and tissue engineering. Similar to collagen, the molecular composition of gelatin comprises of a repeating amino acid sequence of glycine-X-Y triplets, where X and Y are frequently proline and hydroxyproline. Despite its biocompositional similarities, intrinsic bioactivity and biocompatibility, gelatin lacks structural integrity and appropriate mechanical properties matching the collagen-rich host tissues for in vivo applications, which arises due to the inherent differences in its molecular configurations and temperature-dependent phase transitions between gelatin and collagen. Gelatin molecules can adapt to a random coiled configuration with some domains of triple helix configuration of collagen depending on the nature and extent of denaturing process that collagen underwent. Triple helix domains dominate to form aggregates below a temperature of approximately 30° C. (gel structure), however above 30° C. on thermal transition, gelatin molecules prefer a random coil configuration and solubilizes in water. The lack of structural stability limits its application as three-dimensional scaffolds in physiological conditions, which is commonly overcome by crosslinking gelatin molecules that restrict flow on heating. While crosslinking enhances the stiffness of the gelatin scaffolds, it often compromises their ability to expand or strain under minimal stress, i.e. compliance (inverse of stiffness). In addition, due to lack of energy dissipation mechanism, its suture retention strength remains poor.

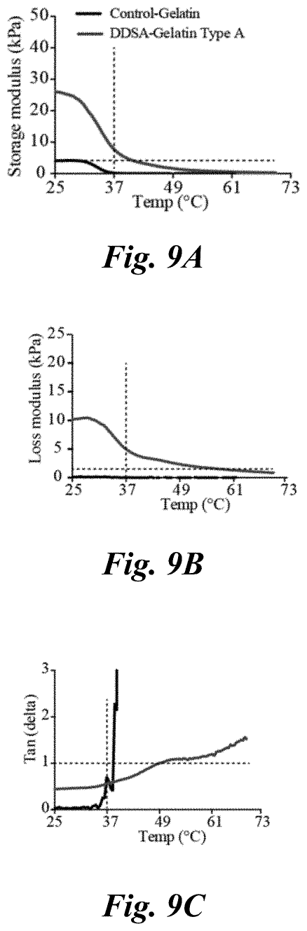

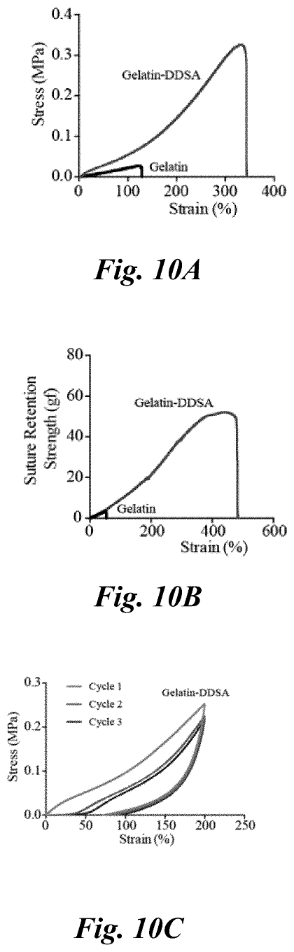

Accordingly, provided herein is a simple, inexpensive, elastin-free gelatin-based material composition to develop elastomeric materials. Gelatin can be modified with aliphatic molecules of various carbon chain lengths (e.g. C₉-C₁₈, including C₉, C₁₀, C₁₁, C₁₂, C₁₃, C₁₄, C₁₅, C₁₆, C₁₇, and C₁₈) to yield a tough, extensible, and suturable material without external crosslinking. For example, referring now to FIGS. 9A-9C and FIGS. 10A-10C, gelatin on modification with an aliphatic molecule, dodecenylsuccinic anhydride (DDSA, chain length: C₁₂) became approximately 2500% more tougher and stretched up to 350% of its initial length compared to only approximately 135% for its control counterpart with in only 0.3 MPa of applied tensile stress. The suture retention strength increased from a very low value of 3-5 gm-force to above 50 gm-force. In addition to hyper-extensibility, the resultant gelatin scaffolds are thermally stable at 37° C. and also can be 3D printed in custom shape and designs.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Langer, R., and Vacanti J. P. Tissue Engineering. Science 260, 920, 1993.
Gilbert, T. W., Sellaro, T. L., and Badylak, S. F. Decellularization of tissues and organs. Biomaterials 27, 3675, 2006.
Parnigotto, P. P., Conconi, M. T., Gamba, P. G., and Midrio P. Experimental defect in rabbit urethra repaired with acellular aortic matrix. Urol Res 16, 28(1), 46, 2000.
Badylak, S. F., Freytes, D. O., and Gilbert, T. W. Extracellular matrix as a biological scaffold material: structure and function. Acta Biomater 5, 1, 2009.
Nakamura, N., Kimura, T., and Kishida A. Overview of the development, applications, and future perspectives of decellularized tissues and organs. ACS Biomater Sci Eng 3, 1236, 2016.
Chang, Y., Tsai, C. C., Liang, H. C., and Sung, H. W, In vivo evaluation of cellular and acellular bovine pericardia fixed with a naturally occurring crosslinking agent (genipin). Biomaterials 23, 2447, 2002.
Sung, H. W., Huang, R. N., Huang, L. L., Tsai, C. C., and Chiu, C. T. Feasibility study of a natural crosslinking reagent for biological tissue fixation, J Biomed Maters Res Part A 42, 560, 1998.
Sung, H. W., Chang, Y., Chiu, C. T., Chen, C. N., and Liang, H. C. Crosslinking characteristics and mechanical properties of a bovine pericardium fixed with a naturally occurring crosslinking agent J Biomed Maters Res Part A 47, 116, 1999.
Bhrany, A. D., Lien, C. J., Beckstead, B. L., Futran, N. D., Muni, N. H., Giachelli, C. M., and Ratner, B. D. Crosslinking of an oesophagus acellular matrix tissue scaffold. J Tissue Eng Regen Med 2, 365, 2008.
Koo, H. J., Lim, K. H., Jung, H. J., and Park, E. H. Anti-inflammatory evaluation of gardenia extract, geniposide and genipin. J Ethnopharmacol 103, 496, 2006.
Suzuki, Y., Kondo, K., Ikeda, Y., and Umemura, K. Antithrombotic effect of geniposide and genipin in the mouse thrombosis model. Planta Med 67, 807, 2001.
Haag, J., Baiguera, S., Jungebluth, P., Barale, D. Del, G. C., Castiglione, F., Bianco, A., Comin, C. E., Ribatti, D, and Macchiarini, P. Biomechanical and angiogenic properties of tissue-engineered rat trachea using genipin cross-linked decellularized tissue. Biomaterials 33, 780, 2012.
Okada, K, Shoda, J., Kano, M., Suzuki, S., Ohtake, N., Yamamoto, M., Takahashi, H., Utsunomiya, H., Oda, K., Sato, K., and Watanabe, A. Inchinkoto, a herbal medicine, and its ingredients dually exert Mrp2/MRP2-mediated choleresis and Nrf2-mediated antioxidative action in rat livers. Amer J Physiol-Gastrointest Liver Physiol 292, G1450, 2007.
Sun. F., Jiang. Y., Xu, Y., Shi, H., Zhang. S., Liu, X., Pan, S., Ye, G., Zhang, W., Zhang, F., and Zhong, C. Genipin cross-linked decellularized tracheal tubular matrix for tracheal tissue engineering applications. Sci Rep 6, 2016.
Gilbert, S. M., Lai, J., Saigal, C. S., and Gore, J. L. Urologic Diseases in America Project. J Urol 190, 916, 2013.
Stein, R., Schroder, A., and Thuroff, J. W. Bladder augmentation and urinary diversion in patients with neurogenic bladder: surgical considerations. J Pediatr Urol 8, 153, 2012.
Shimko, M. S., Tollefson, M. K., Umbreit, E. C., Farmer, S. A., Blute, M. L, and Frank, I. J Urol 185, 562, 2011.
Van der Aa, F., Joniau, S., Van Den Branden, M., and Van Poppel, H. Metabolic changes after urinary diversion. Adv Urol 764325, 2011.

McDougal, W. S. Metabolic complications of urinary intestinal diversion. J Urol 147, 1199, 1992.

Ferriero, M., Guaglianone, S., Papalia, R., Muto, G. L., Gallucci, M., and Simone, G. Risk assessment of stone formation in stapled orthotopic ileal neobladder. J Urol 193, 891, 2015.
Okhunov, Z., Duty, B., Smith, A. D. and Okeke, Z. Management of urolithiasis in patients after urinary diversions. BJU Int 108, 330, 2011.
Horst, M., Madduri, S., Goblet, R., Sulser, T., Milleret, V., Hall, H., Atala, A., and Eberli, D. Engineering functional bladder tissues. J Tissue Eng Regen Med 7, 515, 2013.
de Kemp, V., de Graaf, P., Fledderus, J. O., Ruud Bosch, J. L., and de Kort, L. M. Tissue engineering for human urethral reconstruction: systematic review of recent literature. PloS one 10, e0118653, 2015.
Kates, M., Singh, A., Matsui, H., Steinberg, G. D., Smith, N. D., Schoenberg, M. P., and Bivalacqua, T. J. Curr Urol Rep 16, 8, 2015.
Sopko, N. A., Kates, M., and Bivalacqua, T. J. Use of regenerative tissue for urinary diversion. Curr Opin Urol 25, 578, 2015.
Pokrywczynska, M., Adamowicz, J., Sharma, A. K., and Drewa, T. Human urinary bladder regeneration through tissue engineering—an analysis of 131 clinical cases. Exp Biol Med (Maywood) 239, 264, 2014.
Atala, A., Bauer, S. B., Soker, S., Yoo, J. J., and Retik, A. B. Tissue-engineered autologous bladders for patients needing cystoplasty. Lancet 367, 1241, 2006.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a modified collagen or gelatin, wherein an amine of at least one or more lysine or hydroxylysine residues of the collagen or gelatin is covalently bond to a C₂ to C₁₈ substituted or unsubstituted, saturated or unsaturated aliphatic hydrocarbon chain.

2. The composition of claim 1, wherein the modified collagen or gelatin has one or more repeating units comprising the following moiety: 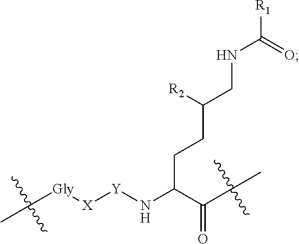
wherein:
Gly is glycine;
X and Y can be the same or different and are each amino acids;
R₁ is selected from the group consisting of: —CH₂—CₙH₂ₙ₊ₚ and —CH₂—CH(CH₂C(O)OH)—CH₂—CH═CH—CₙH₂ₙ₊ₚ; wherein n is an integer selected from the group consisting of 2, 3, 4, 5, 6, 7, 8, 9, 10, 11, 12, 13, 14, 15, 16, 17, and 18; and p is 0 or 1; and
R₂ is selected from the group consisting of H, —OR₃, —NH₂, C₁-C₄ alkyl, —CF₃, and —COOR₄, wherein R₃ and R₄ are each independently H or C₁-C₄ alkyl.

3. The composition of claim 2, wherein X is proline.

4. The composition of claim 2, wherein Y is selected from the group consisting of hydroxyproline, lysine, and hydroxylysine.

5. The composition of claim 2, comprising one or more repeating units comprising the following moiety:

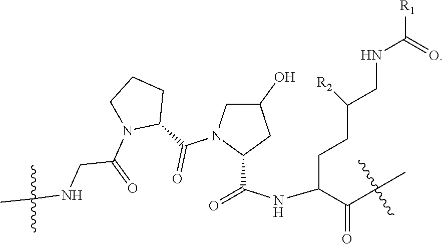

6. The composition of claim 5, comprising one or more repeating units selected from the group consisting of:

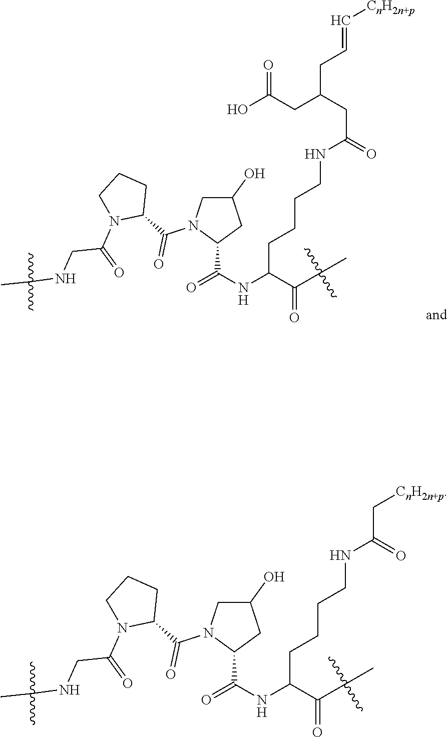

7. The composition of claim 7, wherein p is 1 and n is selected from the group consisting of 9, 10, 11, and 12.

8. The composition of any of claims 1-7, wherein the composition is about 250% tougher than control collagen or gelatin.

9. The composition of any of claims 1-7, wherein the composition can be reversibly stretched up to about 450% of an initial dimension upon application of about 0.2 MPa force per unit area.

10. The composition of any of claims 1-7, wherein the composition can withstand suture retention loads greater than about 50 gram-force.

11. The composition of claim 10, wherein the composition can withstand suture retentions loads up to about 350 gram-force.

12. A tissue-engineering scaffold comprising the composition of any of claims 1-7.

13. The tissue-engineering scaffold of claim 12, wherein the scaffold supports in vitro growth of human smooth muscle cells.

14. The tissue-engineering scaffold of claim 13, therein the human smooth muscle cells are selected from the group consisting of genitourinary cells, gastrointestinal cells, heart cells, blood vessel cells, and skin cells.

15. An implant comprising the composition of any of claims 1-7.

16. A method for preparing a modified collagen or gelatin, the method comprising contacting a collagen or gelatin sample with a solution comprising a C₂-C₁₈ succinic anhydride or C₂-C₁₈ N-hydroxysuccinimide ester and a suitable reagent for a period of time to form a modified collagen or gelatin.

17. The method of claim 16, wherein the suitable reagent is a tertiary amine.

18. The method of claim 17, wherein the tertiary amine is 4-(dimethylamino)pyridine (DMAP).

19. The method of claim 16, wherein the collagen is a vitrified collagen.

20. The method of claim 16, wherein the collagen is not a vitrified collagen.

21. The method of claim 16, further comprising neutralizing the collagen.

22. The method of claim 16, wherein the collagen is selected from the group consisting of collagen I, collagen II, collagen III, and collagen X.

23. The method of claim 22, wherein the collagen is collagen type I.
